(19) United States
(12) Patent Application Publication
(10) Pub. No.: US 2010/0036310 A1
(43) Pub. Date: Feb. 11, 2010

(54) INTEGRATED PATIENT MANAGEMENT AND CONTROL SYSTEM FOR MEDICATION DELIVERY

(76) Inventor: ROBERT S. HILLMAN, San Diego, CA (US)

Correspondence Address: O''Melveny & Myers LLP;IP&T Calendar Department LA-13-A7, 400 South Hope Street, Los Angeles, CA, 90071-2899, US

(21) Appl. No.: 12/534,447

(22) Filed: Aug. 3, 2009

Related U.S. Application Data
(60) Provisional application No. 61/086,383, filed on Aug. 5, 2008.

(51) Int. Cl. A61M 5/168; A61N 1/30; H04Q 5/22

(52) U.S. Cl. 604/20; 604/66; 340/10.1

(57) ABSTRACT

An integrated patient monitoring and control system is provided which includes a sample set, the sample set being adapted for coupling to the patient to obtain a specimen from the patient, a sensor, the sensor being adapted to receive the specimen from the sample set and to analyze the sample, a medication control unit, the medication control unit receiving information from the sensor, and utilizing that information to determine medication dosing information for the patient, and a medication administration system, the medication administration system receiving the dosing information from the medication control unit, and adapted to cause administration of the medication to the patient. If the sample set is adapted for blood draw, the system advantageously is performed in conjunction with a pneumatic pressure cuff, inflated so as to aid in blood draw.

### RELATED APPLICATION INFORMATION

This application claims priority to and benefit of U.S. Provisional Application Ser. No. 61/086,383, filed Aug. 5, 2008, entitled “Integrated Patient Management and Control System for Medication Delivery” (Our Reference 037,028-002), the content of which is incorporated by reference herein in its entirety as if fully set forth herein.

This application is related to U.S. Provisional Application Ser. No. 61/139,826, filed Dec. 22, 2008 (Our Reference 037,028-003); U.S. Provisional Application Ser. No. 61/171,904, filed Apr. 23, 2009 (Our Reference 037,028-004); and U.S. Provisional Ser. No. 61/172,433, filed Apr. 24, 2009 (Our Reference 037,028-005), each of which are incorporated herein by reference in their entirety as if fully set forth herein.

### FIELD OF THE INVENTION

The invention relates generally to an automated closed loop (feedback controlled) drug delivery system using a optimal sampling method and control system. More particularly, the invention relates to methods and apparatus for use in the administration of drugs, such as heparin as an anti-coagulant medicine used in the treatment of cardiovascular and neurovascular disease as well as deep-vein thrombosis and pulmonary embolic disease.

### BACKGROUND OF THE INVENTION

Millions of patients are treated with unfractionated heparin (UFH) in the acute care hospital setting to control their level of anticoagulation. These patients are monitored by a multi-step, labor intensive process to maintain their level of anticoagulation. This complex process leads to frequent human error, thus only 35%-50% of patients are within a safe range of heparin at any given time. The consequences of both under- and over-anticoagulation include death, heart attack, stroke, moderate to severe blood loss, tremendous strain on the patient and their loved ones, and millions dollars in avoidable health care costs. The problem has become so serious that the Joint Commission recently issued a “Sentinel Event Alert”¹ regarding the prevention of errors related to heparin. Such alerts require immediate investigation and response for an event that carries a significant chance of a serious adverse outcome. Several approaches have tried to improve control of heparin levels. These approaches include point-of-care monitoring and use of standardized nomograms. The attempts have yielded little if any improvement.

Heparin, alone or in conjunction with other antithrombotic agents, is the standard of treatment in patients with acute myocardial infarction (AMI), unstable angina (UA), thrombosis, deep vein thrombosis, or pulmonary embolism. Heparin produces a dose-dependent prolongation of the clotting process measured by the activated partial thromboplastin time (aPTT). However, the anticoagulant effects of heparin are variable. Previous studies have reported wide subject variation in the dose of heparin required to achieve and maintain a therapeutic aPTT². A study, published in February 2009 in Circulation,³ further confirmed that only 33% of patients receiving heparin had therapeutic anticoagulation. The consequences of too high or too low a level of anticoagulation can be serious.⁴ In patients with acute ischemic syndromes, inadequate anticoagulation may lead to recurrent thrombosis, and significant bleeding has occurred in patients at supra-therapeutic doses of heparin. When a fixed dose of heparin is used as conjunctive therapy to thrombolysis or in the treatment of AMI, a substantial percentage of patients can be above or below the aPTT therapeutic range at any point in time.

Heparin is a naturally-occurring anticoagulant that when administered intravenously prevents the formation of clots and extension of existing clots within the blood. It is used for a number of different conditions. It is given as a continuous infusion for management of acute coronary syndromes, stroke, pulmonary emboli and venous thrombosis. Since the goal of therapy is to achieve a target range of anticoagulation rapidly and then maintain that level for a period of time, continuous infusions are monitored periodically and the dose is adjusted. Heparin dosing is complicated by the illness itself monitoring heparin pharmacokinetics. Thus, in the acute phase of a major thrombosis, heparin half-life is shorter than after a period of heparin treatment. Thus monitoring and dose adjustment are required to optimize therapy primarily for anticoagulation for cardiovascular conditions, including acute coronary syndromes, myocardial infarction, atrial fibrillation, cardiopulmonary bypass surgery (CABG), percutaneous coronary intervention (PCI), deep vein thrombosis and pulmonary embolism.

In the administration of heparin, the objective is to achieve an activated partial thromboplastin time (aPTT) value calculated based on the patient's aPTT. As a result of the difficulty to correctly titrate heparin to any given patient, on average only 30% to 40% of patients achieve the desired aPTT range +/−15 seconds of administration during the course of therapy.⁵

The worldwide market for unfractionated heparin is estimated at $400 million.⁶ The US market for unfractionated heparin is about $146 million. It is a generic drug with Baxter, APP and Hospira comprising 80% of the market.⁷ Sales of heparin have maintained a steady growth over the past few years. From June 2006 to June 2007, total US heparin sales units grew by 6%.⁸ With the recent Baxter heparin recall early in 2008, the market (unit sales) has declined slightly as a result of less supply available in the market; however with manufacturers such as APP increasing production capacity, heparin supply should recover within the year.

Heparin is associated with many medication errors as a result of its complex pharmacologic response and large inter-patient variability in response. According to the United States Pharmacopoeia (USP) MED-MARX⁹, during a five year period from 2003 to 2007, heparin medication errors totaled 17,000 out of more than 50,000 anticoagulation related medication errors.¹⁰ The majority of heparin errors occur during administration at the bedside (47.6%) followed by prescribing errors (14.1%), dispensing (13.9%) and transcribing and documenting (18.8%). A majority of these errors resulted from a failure to follow procedures and protocols.¹¹

Close monitoring of patients on heparin is extremely important: too low a dose of heparin can lead to under anticoagulation while too high a dose can lead to serious bleeding. It is also important to bring patients into range as quickly as possible to avoid adverse outcomes.¹² In studies of patients with acute coronary syndromes treated with intravenous heparin, increasing aPTT values were associated with increased bleeding episodes.¹³ At various times throughout therapy, only 50% of patients had aPTT values in the therapeutic range.¹⁴

Lower than required dosing levels of heparin can lead to episodes of thromboembolic complications in patients with acute coronary syndromes (ACS) or deep vein thrombosis while higher than required levels of heparin can lead to bleeding complications.¹⁵ In the recent “Can Rapid Risk Stratification of Unstable Angina Patients Suppress Adverse Outcomes with Early Implementation of the American College of Cardiology/American Heart Association Guideline (CRUSADE) initiative, it was observed that 49% of patients received excess dosing of unfractionated heparin leading to a significantly higher rate of major bleeding and need for transfusion as compared to patients who did not receive excess dosing.¹⁶

The problem has become so serious that the Joint Commission, which accredits all US hospitals issued a “Sentinel Event Alert”¹⁷ regarding the prevention of errors related to commonly used anticoagulants. Such alerts signal the need for immediate investigation and response for an event that carries a significant chance of a serious adverse outcome.

Current practices for the administration of heparin in an acute care setting involve many different steps and resources that can easily tax the hospital staff and lead to human error. General heparin dosing protocols (nomograms) may include the following steps: a standard initial bolus of heparin with a calculated infusion rate normally based on the patient's weight; instructions for drawing partial thromboplastin time (aPTT) and orders for dosing adjustments in response to measured aPTT and other values. The nurse will take a blood sample and send it to the central lab for analysis. The lab will provide the result to the nurse and the nurse will then evaluate the result and make the necessary adjustments to the dose based on the results. The nurse will check with the physician to verify dosing. Upon receiving approval from the physician, the nurse will make the necessary adjustment to the infusion rate. This process requires at least 1-2 hours to complete each time and is repeated every 4 to 6 hours over the course of approximately 2.5 days while the patient is receiving heparin. FIG. 1 shows a work flow for heparin monitoring.

As medication errors have continued to occur with heparin, sometimes causing serious complications, many hospitals and organizations have devised ways to try to minimize medication errors. Besides instituting nomograms for heparin administration, hospitals have tried other systems such as bar coding software that can identify and verify the drug and its concentration; inpatient anticoagulation services for heparin in which pharmacists run the services that provide daily pharmacy input on dosing and monitoring for patients on heparin; and automated medication dispensing systems.

The introduction of “smart” infusion pumps in the past few years has tried to address the issue of dosing errors before the patient suffers any negative effects. These smart pumps, which are still only used in approximately 50% of all hospitals in the US¹⁸, contain comprehensive drug libraries and standardized dosing units based on the specific acute care area of use. They also have dose calculators and alert systems if dosing falls out of pre-determined parameters. Nevertheless, recent reviews have concluded that many users of smart pumps bypass the safety features of the devices, and as a result medication errors continue to occur.¹⁹

Smart pumps simply avoid the nurse from inadvertently typing in a dose outside the standard dosing range. There is no provision for individualizing the dose for each patient, nor is there the ability to use a test measure of patient response to adjust dosing. For medications with variable patient response (e.g. unfractionated heparin, insulin) the use of more individualized dosing and individualized adjustment according to a blood test has the potential to advance therapy and improve response.

Hospitals are increasingly concerned about medication errors. They are also in search of tighter control of critical parameters in the ICU, including anticoagulation and blood glucose. As a result, there is significant opportunity for a smart-controller that can integrate critical diagnostic assays and information to adjust patient dosing safely. With renewed focus on eliminating human error in drug administration of potent intravenous agents in the hospital, there is a large unmet need. While previous systems have been described, see, e.g., Hillman et al., “Feedback Controlled Drug Delivery System”, U.S. Pat. No. 5,697,899, issued Dec. 16, 1997, Valcke et al., “Method and Apparatus For Closed Loop Drug Delivery”, U.S. Pat. No. 5,733,259, issued Mar. 31, 1998 and Gauthier et al., “Feedback Controlled Drug Delivery System”, U.S. Pat. No. 6,017,318, issued Jan. 25, 2000, they do not contain or integrate all of the advanced features in the current invention that are designed to further minimize medication errors and further improve the level of control.

### SUMMARY OF THE INVENTION

An integrated patient monitoring and control system is provided which includes a sample set, the sample set being adapted for coupling to the patient to obtain a specimen from the patient, a sensor, the sensor being adapted to receive the specimen from the sample set and to analyze the sample, a medication control unit, the medication control unit receiving information from the sensor, and utilizing that information to determine medication dosing information for the patient, and a medication administration system, the medication administration system receiving the dosing information from the medication control unit, and adapted to cause administration of the medication to the patient. In one embodiment, the sample set is adapted for blood draw from the patient. Advantageously, the blood draw is performed in conjunction with a pneumatic pressure cuff, inflated so as to aid in blood draw.

In another embodiment, a multi-parameter integrated patient monitoring and control system includes a sample set, the sample set being adapted for coupling to the patient to obtain a specimen from the patient, a sensor, the sensor being adapted to receive the specimen from the sample set and to analyze the sample, the sensor including a first assay and at least a second assay, the assays testing for different medical conditions or different drugs, a medication control unit, the medication control unit receiving information from the sensor including information on the first and second assay, and utilizing that information to determine medication dosing information for the patient, and a medication administration system, the medication administration system receiving the dosing information from the medication control unit, the system including a first drug to be administered corresponding to the first assay and a second drug to be administered corresponding to the second assay, and adapted to cause administration of the medication to the patient. By way of example, the first assay could relate to blood clotting, e.g., aPTT, ACT, or Factor Xa value, and the first drug be heparin, and the second assay could relate to blood glucose level, and the second drug be insulin.

In yet another embodiment, a multi-parameter integrated patient monitoring and control system includes a sample set, the sample set being adapted for coupling to the patient to obtain a specimen from the patient, a sensor, the sensor being adapted to receive the specimen from the sample set and to analyze the sample, a medication control unit, the medication control unit receiving information from the sensor and at least one other patient information parameter, and utilizing that information to determine medication dosing information for the patient, and a medication administration system, the medication administration system receiving the dosing information from the medication control unit, and adapted to cause administration of the medication to the patient. In addition to the results of the first assay (that contains information relating to the patient response to the first drug being administered), a second item of patient information may be information from at least a second sensor or sensors or information relating to a first drug being administered, such as the drug level of the patient or information relating to the pharmacodynamic response of the patient to the first drug. The other patient information may also be the patient's vital signs, such as the blood pressure or heart rate of the patient.

### BRIEF DESCRIPTION OF THE DRAWINGS

FIG. 1 shows the cycle of the sample withdrawal set, the sensor, the medication control unit and the drug delivery technology.

FIG. 2 is a schematic block diagram of the main components of the system.

FIG. 3 is a detailed block diagram of the system.

FIG. 4 is a flowchart showing overall operation of the system.

FIG. 5 shows a perspective view of the integrated patient management and control system for medication delivery.

FIG. 6 shows a perspective view of an alternate embodiment of the integrated patient management and control system for medication delivery.

FIG. 7A shows a top down view of an assay showing alternating assay regions. FIG. 7B shows a top down view of an assay showing four differing assays.

FIG. 8 shows a front view of a representative display system.

### DETAILED DESCRIPTION

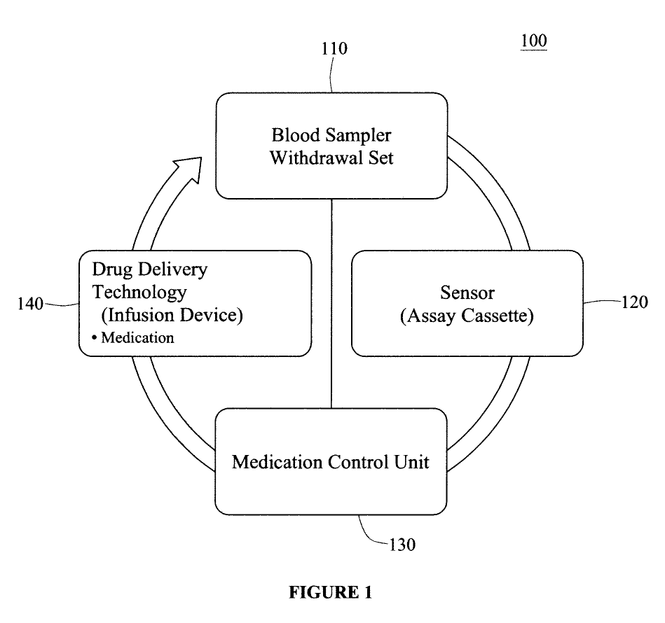

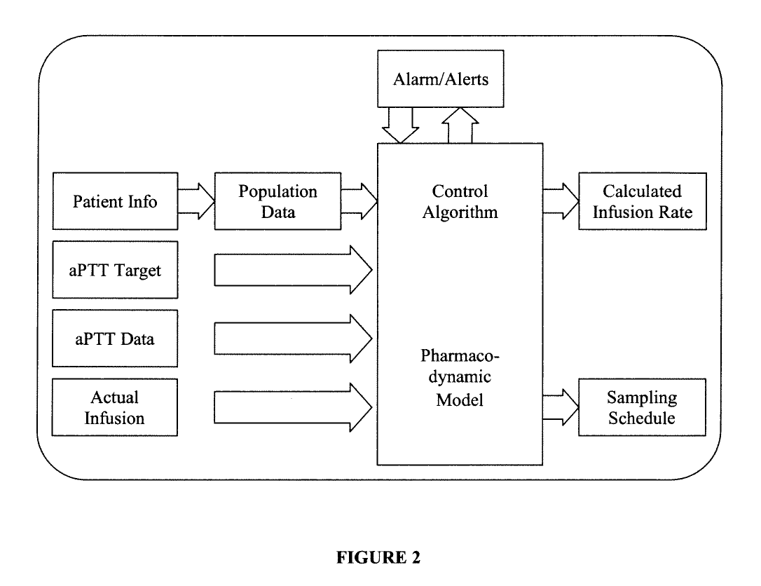

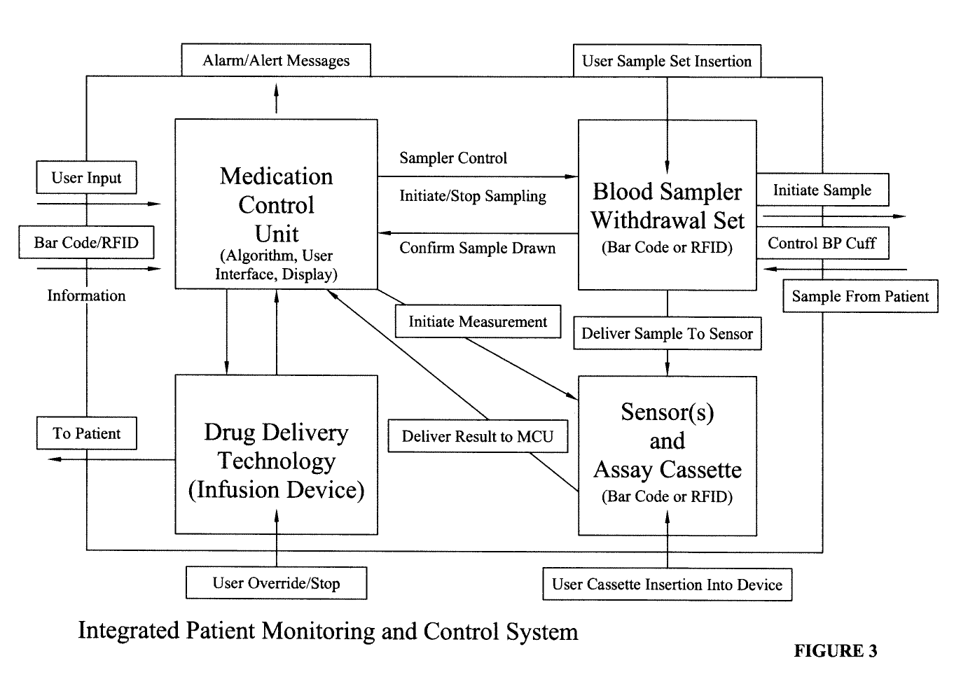

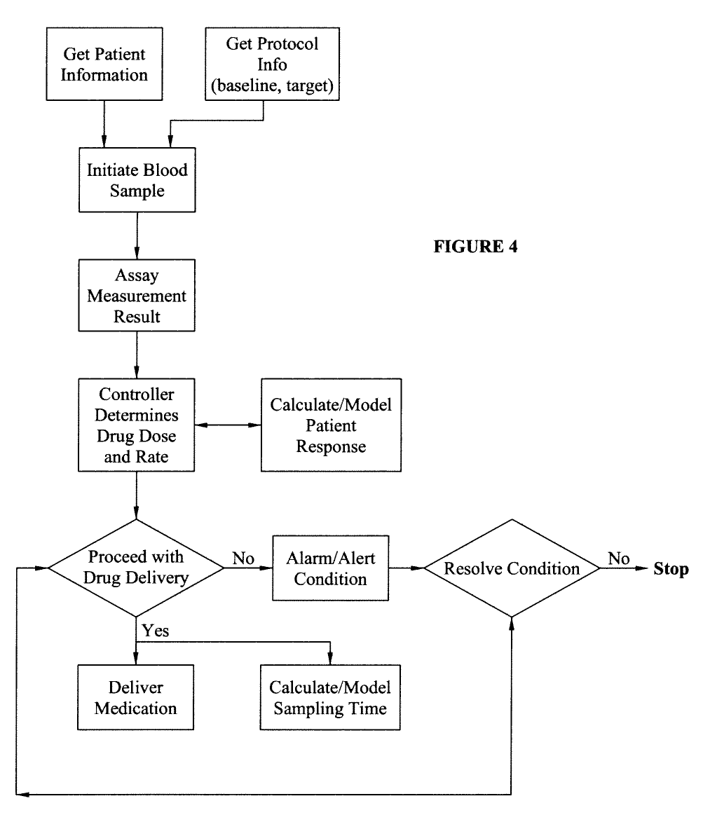

With particular reference to FIGS. 1, 2, 3 and 4, this invention describes an integrated patient measurement and control system 100 (IPMC) for delivering medications. The preferred elements of the system as depicted are the blood sampler/withdrawal set 110, one or more sensors, 120 a medication control unit 130 and an integrated drug delivery technology 140 through which medication can be delivered.

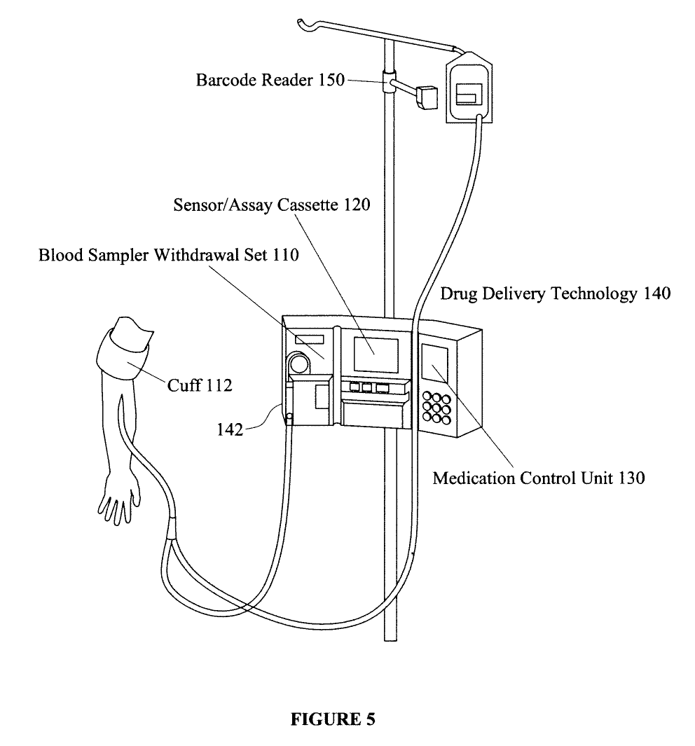

In one aspect, one of the key features of the IPMC System is an Integrated Drug Delivery Technology, shown in FIG. 5 is an integrated intravenous (IV) infusion pump. This integration minimizes the chance for communication errors that could occur with an external infusion device leading to potentially serious consequences such as infusion without proper feedback. Additional elements of the system include an integrated bar code reader 150 to read the name, dosage, and concentration of the medication to be delivered and patient ID to further minimize any medication delivery errors; intermittent sampling and control, and a cuff that can be used in conjunction with the sampler device and medication control unit. The system is capable of controlling different medications via interchangeable sensor and algorithms, or multiple medications through a multiplexed assay cassette.

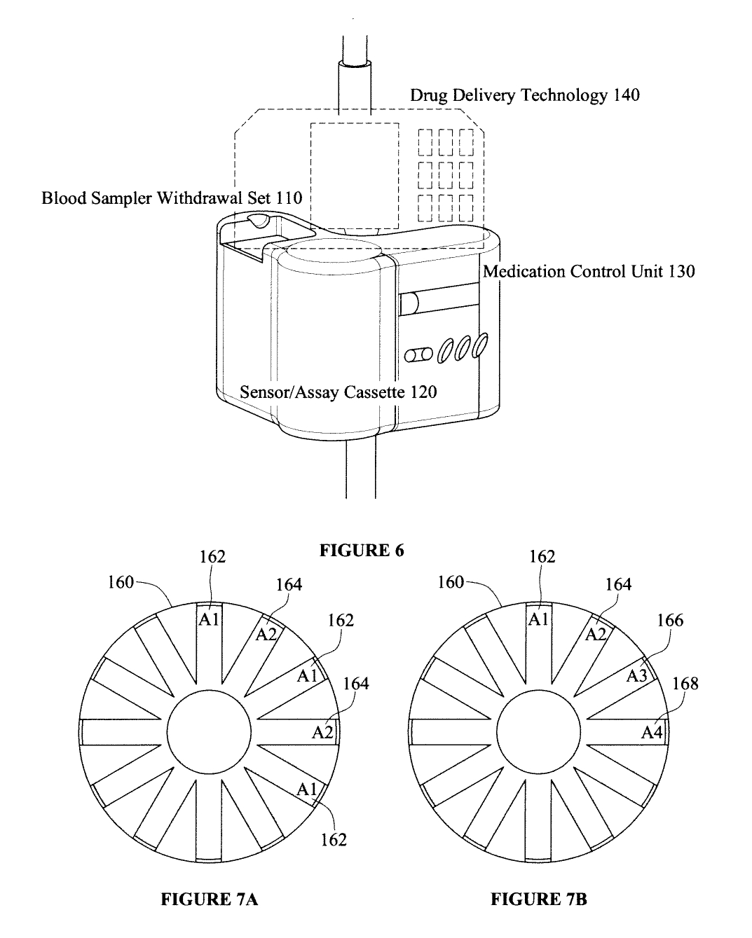

An alternative embodiment of the system is shown in FIG. 6, again containing integration of all of the elements described.

Each of the individual elements of the invention are described below.

### Sampling System/Withdrawal Set

The sampling system can withdrawal any biological fluid including blood, urine, interstitial fluid, or saliva. The preferred sample is blood. The sampling system preferably contains a bar code/RFID tag and interlock with the system to ensure patient safety and notify the medication control unit if any errors occur (e.g. occlusion, attempted removal, etc). The sampling system is capable of either intermittent sampling or could be adapted to continuous sampling based on the sensor(s).

The preferred embodiment of the sampling system incorporates a cuff 112 (blood pressure like cuff) and works in conjunction with the controller and sampler to ensure smooth withdrawal of blood. The sampling system is coupled with a specific algorithm to inflate automatically prior to sampling (an automated corresponding to a tourniquet manually used for a lab blood draw) and use a sensing algorithm to set the pressure just above the systolic pressure to ensure a smooth draw and more frequent success to prevent vein collapse (especially in elderly).

The sampling system is housed in a cassette that will fit into the device. In one aspect of the invention, an interlock system and optionally RFID pair it with the IPMC.

### Feedback Sensor(s)

The IPMC 110 is a modular system with the capability of providing feedback on different parameters from different medications or on more than one parameter (e.g., drug level, pharmacodynamic response) simultaneously. This is achieved by having the sensor be interchangeable in the device or by a sensor that can be used with more than one assay parameter. One embodiment, shown below in FIGS. 7A and 7B, is a cassette 160 which consists of multiple assays for different assays (e.g., a1 162, a2 164 (alternating); or a1 162, a2 164, a3 166, a4 168 (in sequence)). Thereby multiple assay parameters (e.g. aPTT, glucose concentration, potassium level) can be detected in sequence. The embodiment below preferably interlocks with the system and contains a barcode/RFID tag to ensure that the correct parameters are being measured.

In another aspect of the invention of the system, vital signs monitoring (e.g. ECG, blood pressure, Sp02) is integrated into the overall monitoring of the safety and state of patient. The blood pressure and heart rate can be analyzed using the cuff 112 that is part of the sampling system.

### Algorithm and Medication Control Unit (MCU)

The IPMC System is based on intermittent sampling or if the sensor allows, continuous measurement. It is important to note that the sampling system may take intermittent samples, and the MCU 130 uses algorithms to reconstruct patients state, response and then calculate drug delivery rate based on intermittent samples. In addition, the optimal sampling time to take a sample can be determined by response of patient test and if patient response is unexpected (e.g., in wrong direction) the medical delivery is halted.

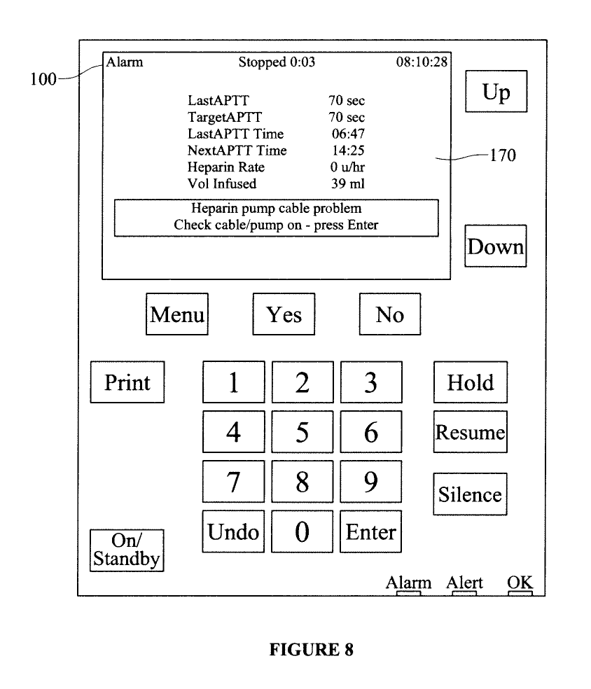

There is also an alarm/alert infrastructure/supervisory system 100 to oversee the entire MCU. If all aspects of the IPMC System are functioning there is a “green light” and delivery proceed. If there is an alert, (e.g., a non-critical problem that is potentially correctable) has been detected (e.g. sampling error, communication error, etc.) a yellow alert and audible alarm occurs. If a serious condition occurs (incorrect infusion rate, multiple missed samples, disconnected line) occurs then the system immediately goes into alarm (red light, audible alarm, communication to central station). FIG. 8 shows a representative display of a monitor 170 for the system.

### Medication Delivery Technology

The medication delivery technology optionally consists of intravenous infusion pumps 142, syringe pumps, implantable pumps, transdermal iontophoretic systems. The preferred embodiment is an intravenous infusion pump. The preferred delivery route is intravenous, but other portals such as intrarterial, transdermal, peritoneal, subcutaneous, or buccal could also be used.

In the preferred embodiment, the pump is an integral part of the system rather than connected by an interface. This prevents any potential safety issues including 1) communication errors between devices, 2) incorrect information being sent between devices, 3) loss of control of device, 4) undetected error that is missed by pump and not detected by the medication control unit.

Optionally, the system, will contain a bar code sensor 150 that can read the identity of the medication being delivered as well as its concentration, and patient for whom it is intended.

### Additional Aspects

The system preferably includes telemetry (either wired via ethernet or like, or wireless like bluetooth or WIFI) to communicate information to central station. The system has the ability to pair the system with the patients instructions to make sure the right patient is being started on the right drug.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

### REFERENCES

¹ The Joint Commission Sentinel Event Alert: Preventing errors relating to commonly used anticoagulants Issue 41, Sep. 24, 2008.
² Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996;93:870-878.
³ Cheng S, Morrow D A, Sloan S, Antman E M, Sabatine M S. Predictors of initial nontherapeutic anticoagulation with unfractionated heparin in ST-segment elevation myocardial infarction. Circulation. 2009 Mar 10; 119(9):1195-202. Epub 2009 Feb. 23.
⁴ Anand et al. Relationship of Activated Partial Thromboplastin Time to Coronary Events and Bleeding in Patients with Acute Coronary Syndrome Who Receive Heparin. Circulation. 2003; 107:2884-2888.
⁵ Cannon et al. Automated Heparin Delivery System to Control Activated Partial Thromboplastin Time. Circulation. 1999;99:751-756.
⁶ Alchemia's generic fondaparinux a potential beneficiary of heparin product recall. Alchemia Ltd. press release: Mar. 27, 2008. <http://www.alchemia.com>
⁷ IMS National Sales Perspective Report. IMS Health Inc. June 2008.
⁸ Ibid.
⁹ MEDMARX® is a national database that tracks and trends adverse drug reactions and medication errors.
10 C. Peterson, C. Ham, T. Vanderveen. Improving Heparin Safety: A Multidisciplinary Invited Conference. Hospital Pharmacy, Vol. 43, No. 6, pp 491-497.
¹¹ Ibid.
¹² Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996;93:870-878.
¹³ Anand et al. Relationship of Activated Partial Thromboplastin Time to Coronary Events and Bleeding in Patients with Acute Coronary Syndrome Who Receive Heparin. Circulation. 2003;107:2884-2888.

¹⁴ Ibid.

¹⁵ T. K. Gandhi et al. Protocols for High-Risk Drugs: Reducing Adverse Drug Events Related to Anticoagulants. Agency for Healthcare Research and Quality (AHRQ).
¹⁶ T Y Wang, E Peterson, M Ohman et al. Excess Heparin Dosing Among Fibrinolytic-treated Patients with ST-Segment Elevation Myocardial Infarction. American Journal of Medicine (2008) 121:805-810.
¹⁷ The Joint Commission Sentinel Event Alert: Preventing errors relating to commonly used anticoagulants Issue 41, Sep. 24, 2008.
¹⁸ C. Peterson, C. Ham, T. Vanderveen. Improving Heparin Safety: A Multidisciplinary Invited Conference. Hospital Pharmacy, Vol. 43, No. 6, pp 491-497.
¹⁹ Smart Pumps Are Not Smart On Their Own. Institute for Safe Medication Practices Newsletter, Apr. 19, 2007.

What is claimed:

1. An integrated patient monitoring and control system comprising:
a sample set, the sample set being adapted for coupling to the patient to obtain a specimen from the patient,
a sensor, the sensor being adapted to receive the specimen from the sample set and to analyze the sample,
a medication control unit, the medication control unit receiving information from the sensor, and utilizing that information to determine medication dosing information for the patient, and
a medication administration system, the medication administration system receiving the dosing information from the medication control unit, and adapted to cause administration of the medication to the patient.

2. The integrated patient monitoring and control system of claim 1 wherein the sample set is adapted for blood draw from the patient.

3. The integrated patient monitoring and control system of claim 2 further including a pneumatic pressure cuff.

4. The integrated patient monitoring and control system of claim 3 wherein the pressure cuff is adapted to aid in the blood draw from the patient.

5. The integrated patient monitoring and control system of claim 4 wherein the cuff is inflated above the systolic blood pressure.

6. The integrated patient monitoring and control system of claim 3 wherein the pressure cuff is adapted to measure vital signs of the patient.

7. The integrated patient monitoring and control system of claim 6 wherein the vital signs include the blood pressure of the patient.

8. The integrated patient monitoring and control system of claim 6 wherein the vital signs include the heart rate of the patient.

9. The integrated patient monitoring and control system of claim 1 wherein the sensor measures the coagulation state of the patient's blood.

10. The integrated patient monitoring and control system of claim 9 wherein the sensor measures aPTT.

11. The integrated patient monitoring and control system of claim 9 wherein the sensor measures ACT.

12. The integrated patient monitoring and control system of claim 9 wherein the sensor measures anti-Xa value.

13. The integrated patient monitoring and control system of claim 1 wherein the sensor measures the glucose concentration for the patient sample.

14. The integrated patient monitoring and control system of claim 1 wherein the sensor measures the potassium concentration for the patient sample.

15. The integrated patient monitoring and control system of claim 1 wherein the sensor comprises a carousel.

16. The integrated patient monitoring and control system of claim 15 wherein the carousel includes multiple assays.

17. The integrated patient monitoring and control system of claim 1 wherein the sensor comprises a cassette.

18. The integrated patient monitoring and control system of claim 17 wherein the carousel includes multiple assays.

19. The integrated patient monitoring and control system of claim 1 wherein the sensor includes assays for at least two different physiological states for the patient sample.

20. The integrated patient monitoring and control system of claim 19 wherein the assays for the different physiological states are multiplexed.

21. The integrated patient monitoring and control system of claim 1 wherein the sensor includes an interlock with the sample set.

22. The integrated patient monitoring and control system of claim 1 wherein the sensor has positive communication with the sample set.

23. The integrated patient monitoring and control system of claim 1 wherein the sensor includes a bar code system.

24. The integrated patient monitoring and control system of claim 23 wherein the bar code system provides information regarding contents of the sensor.

25. The integrated patient monitoring and control system of claim 1 wherein the sensor includes a radio frequency identification (RFID) system.

26. The integrated patient monitoring and control system of claim 25 wherein the RFID system provides information regarding contents of the sensor.

27. The integrated patient monitoring and control system of claim 1 wherein the medication control unit conducts intermittent sampling.

28. The integrated patient monitoring and control system of claim 1 wherein the medication control unit conducts continuous sampling.

29. The integrated patient monitoring and control system of claim 1 wherein the medication control unit specifies the sample interval.

30. The integrated patient monitoring and control system of claim 1 wherein the medication control unit specifies the optical sampling interval.

31. The integrated patient monitoring and control system of claim 1 wherein the medication administration unit includes an IV infusion pump.

32. The integrated patient monitoring and control system of claim 1 wherein the medication administration unit includes an intravenous syringe pump.

33. The integrated patient monitoring and control system of claim 1 wherein the medication administration unit includes an implantable infusion pump.

34. The integrated patient monitoring and control system of claim 1 wherein the medication administration unit includes a transdermal iontophoretic drug delivery unit.

35. The integrated patient monitoring and control system of claim 1 wherein the medication administration unit includes a bar code system.

36. The integrated patient monitoring and control system of claim 35 wherein the bar code system provides information regarding name of the drug.

37. The integrated patient monitoring and control system of claim 35 wherein the bar code system provides information regarding dosage of the drug.

38. The integrated patient monitoring and control system of claim 35 wherein the bar code system provides information regarding concentration of the drug.

39. The integrated patient monitoring and control system of claim 35 wherein the bar code system provides information regarding patient information.

40. The integrated patient monitoring and control system of claim 1 wherein the medication administration unit includes a radio frequency identification (RFID) system.

41. The integrated patient monitoring and control system of claim 40 wherein the RFID system provides information regarding name of the drug.

42. The integrated patient monitoring and control system of claim 40 wherein the RFID system provides information regarding dosage of the drug.

43. The integrated patient monitoring and control system of claim 40 wherein the RFID system provides information regarding concentration of the drug.

44. The integrated patient monitoring and control system of claim 40 wherein the RFID system provides information regarding patient information.

45. The integrated patient monitoring and control system of claim 1 further including a drug reversal system capable of overriding the effects of the initial drugs.

46. The integrated patient monitoring and control system of claim 1 wherein the sample set is adapted for urine draw from the patient.

47. The integrated patient monitoring and control system of claim 1 wherein the sample set is adapted for interstitial fluid draw from the patient.

48. The integrated patient monitoring and control system of claim 1 wherein the sample set is adapted for saliva draw from the patient.

49. The integrated patient monitoring and control system of claim 1 further including an alert system.

50. The integrated patient monitoring and control system of claim 1 further including an alarm system.

51. A multi-parameter integrated patient monitoring and control system comprises
a sample set, the sample set being adapted for coupling to the patient to obtain a specimen from the patient,
a sensor, the sensor being adapted to receive the specimen from the sample set and to analyze the sample, the sensor including a first assay and at least a second assay, the assays testing for different medical conditions,
a medication control unit, the medication control unit receiving information from the sensor including information on the first and second assay, and utilizing that information to determine medication dosing information for the patient, and
a medication administration system, the medication administration system receiving the dosing information from the medication control unit, the system including a first drug to be administered corresponding to the first assay and a second drug to be administered corresponding to the second assay, and adapted to cause administration of the medication to the patient.

52. The multi-parameter integrated patient monitoring and control system of claim 51 wherein the first assay relates to blood clotting.

53. The multi-parameter integrated patient monitoring and control system of claim 52 wherein the first assay measures aPTT.

54. The multi-parameter integrated patient monitoring and control system of claim 52 wherein the first assay measures ACT.

55. The multi-parameter integrated patient monitoring and control system of claim 52 wherein the first assay measures factor Xa value.

56. The multi-parameter integrated patient monitoring and control system of claim 52 wherein the first drug is heparin.

57. The multi-parameter integrated patient monitoring and control system of claim 52 wherein the second assay relates to blood glucose level.

58. The multi-parameter integrated patient monitoring and control system of claim 57 wherein the second drug is insulin.

59. A multi-parameter integrated patient monitoring and control system comprising:
a sample set, the sample set being adapted for coupling to the patient to obtain a specimen from the patient,
a sensor, the sensor being adapted to receive the specimen from the sample set and to analyze the sample,
a medication control unit, the medication control unit receiving information from the sensor and at least one other patient information parameter, and utilizing that information to determine medication dosing information and sampling intervals for the patient, and
a medication administration system, the medication administration system receiving the dosing information from the medication control unit, and adapted to cause administration of the medication to the patient at intervals determined by the system.

60. The multi-parameter integrated patient monitoring and control system of claim 59 wherein the other patient information is information from a second sensor.

61. The multi-parameter integrated patient monitoring and control system of claim 59 wherein the other patient information is information relating to a first drug.

62. The multi-parameter integrated patient monitoring and control system of claim 61 wherein the information relating to the first drug is the drug level of the patient.

63. The multi-parameter integrated patient monitoring and control system of claim 61 wherein the information relating to the first drug is the pharmacodynamic response of the patient to the first drug.

64. The multi-parameter integrated patient monitoring and control system of claim 59 wherein the other patient information is information relating to the patient's vital signs.

65. The integrated patient monitoring and control system of claim 64 wherein the vital signs include the blood pressure of the patient.

66. The integrated patient monitoring and control system of claim 64 wherein the vital signs include the heart rate of the patient.
